(19) United States
(12) Patent Application Publication
(10) Pub. No.: US 2018/0330802 A1
(43) Pub. Date: Nov. 15, 2018

(54) ADAPTIVE PATIENT QUESTIONNAIRE GENERATION SYSTEM AND METHOD

(71) Applicant: KONINKLIJKE PHILIPS N.V., Eindhoven (NL)

(72) Inventors: Reza SHARIFI SEDEH, Malden, MA (US); Amir Mohammad TAHMASEBI MARAGHOOSH, Arlington, MA (US)

(21) Appl. No.: 15/978,317

(22) Filed: May 14, 2018

Related U.S. Application Data
(60) Provisional application No. 62/506,209, filed on May 15, 2017.

(51) Int. Cl. G16H 10/20; G16H 10/60; G06N 7/00; G06N 99/00

(52) U.S. Cl. CPC G16H 10/20; G06N 99/005; G06N 7/005; G16H 10/60

(57) ABSTRACT

The present system is configured to generate adaptive, optimal, intelligent questionnaires for diagnosing depression, ADHD, and/or other medical conditions. The system is configured to train a prediction model based on a database of previously asked and answered questions related to various medical conditions. A questionnaire for a specific patient is determined from the questions in the database based on caregiver supplied criteria including a number of questions the questionnaire should have and a quality metric indicating a desired level of relatedness of the questions in the questionnaire to the patient's medical condition. If the patient decides to not answer one or more questions for any reason, another subset of questions are suggested.

### CROSS-REFERENCE TO PRIOR APPLICATIONS

This application claims the benefit of U.S. Provisional Application No. 62/506,209, filed on 15 May 2017. This application is hereby incorporated by reference herein.

### BACKGROUND

### 1. Field

The present disclosure pertains to a system and method for generating an adaptive questionnaire.

### 2. Description of the Related Art

Questionnaire-derived measures related to disease activity and/or characteristics of an individual have been proven to be reliable in the assessment of the individual. As a result of advances in technology and a growing number of computer users, development of computer programs configured to generate questionnaires has expanded. Although typical computer questionnaire systems allow caregivers (or other users) to more easily generate and provide questionnaires to individuals, such questionnaire systems generally either require an individual to answer every question in a questionnaire or provide no follow-up alternative questions to address unanswered questions. The former may cause the individual to provide untruthful or careless answers (e.g., especially if the questionnaire or a given question is lengthy or the individual is uncomfortable with a given question), and the latter may fail to address aspects of the questionnaire covered by unanswered questions, both of which may bias any diagnostic information generated based on the questionnaire. Additionally or alternatively, typical computer questionnaire systems (i) may not enable a caregiver (or other user) to specify how related questions should be to a specific medical condition or other feature related to the individual or (ii) may not implement measures to reduce negative effects to the questionnaire by a questionnaire editor (e.g., the caregiver or other user) when adding/removing questions to/from the questionnaire. These and other drawbacks exist.

### SUMMARY

Accordingly, one or more aspects of the present disclosure relate to an adaptive questionnaire generation system. The system comprises one or more hardware processors and/or other components. The one or more hardware processors are configured by machine readable instructions to receive caregiver expectation criteria for a questionnaire related to a medical condition of a patient. The caregiver expectation criteria comprise (i) information related to a number of questions for the questionnaire and (ii) a quality metric threshold level that indicates a desired relatedness of the questionnaire to the medical condition. The one or more hardware processors are configured to use a prediction model to determine sets of possible questions for forming the questionnaire such that each of the sets of possible questions satisfies the caregiver expectation criteria. Each of the sets of possible questions has classifiers indicating subject matter categories for questions in the respective set of possible questions, and one or more quality metric values that indicate relatedness of the respective set of questions to the medical condition. The one or more quality metric values are determined based on the classifiers and/or other information. The one or more processors are configured to select a set of questions having a fewest number of questions that satisfies the quality metric threshold level from the sets of possible questions for presentation to the patient. The one or more hardware processors are configured to cause presentation of the selected set of questions to the patient.

Another aspect of the present disclosure relates to a method for generating an adaptive questionnaire with a generation system. The system comprises one or more hardware processors configured by machine readable instructions and/or other components. The method comprises receiving, with the one or more hardware processors, caregiver expectation criteria for a questionnaire related to a medical condition of a patient. The caregiver expectation criteria comprise (i) information related to a number of questions for the questionnaire and (ii) a quality metric threshold level that indicates a desired relatedness of the questionnaire to the medical condition. The method comprises using a prediction model to determine, with the one or more hardware processors, sets of possible questions for forming the questionnaire such that each of the sets of possible questions satisfies the caregiver expectation criteria. Each of the sets of possible questions has classifiers indicating subject matter categories for questions in the respective set of possible questions, and one or more quality metric values that indicate relatedness of the respective set of questions to the medical condition, the one or more quality metric values determined based on the classifiers. The method comprises selecting, with the one or more hardware processors, a set of questions having a fewest number of questions that satisfies the quality metric threshold level from the sets of possible questions for presentation to the patient. The method comprises causing, with the one or more hardware processors, presentation of the selected set of questions to the patient.

Still another aspect of present disclosure relates to a system configured for generating an adaptive questionnaire. The system comprises means for receiving caregiver expectation criteria for a questionnaire related to a medical condition of a patient. The caregiver expectation criteria comprise (i) information related to a number of questions for the questionnaire and (ii) a quality metric threshold level that indicates a desired relatedness of the questionnaire to the medical condition. The system comprises means for using a prediction model to determine sets of possible questions for forming the questionnaire such that each of the sets of possible questions satisfies the caregiver expectation criteria. Each of the sets of possible questions has classifiers indicating subject matter categories for questions in the respective set of possible questions, and one or more quality metric values that indicate relatedness of the respective set of questions to the medical condition. The one or more quality metric values are determined based on the classifiers. The system comprises means for selecting a set of questions having a fewest number of questions that satisfies the quality metric threshold level from the sets of possible questions for presentation to the patient; and means for causing presentation of the selected set of questions to the patient.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 illustrates an adaptive questionnaire generation system.

FIG. 2 illustrates example operations performed by the system.

FIG. 3 illustrates a skipped and/or otherwise unanswered questionnaire question and additional alternative questions presented to a patient.

FIG. 4 illustrates a method for generating an adaptive questionnaire with a generation system.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

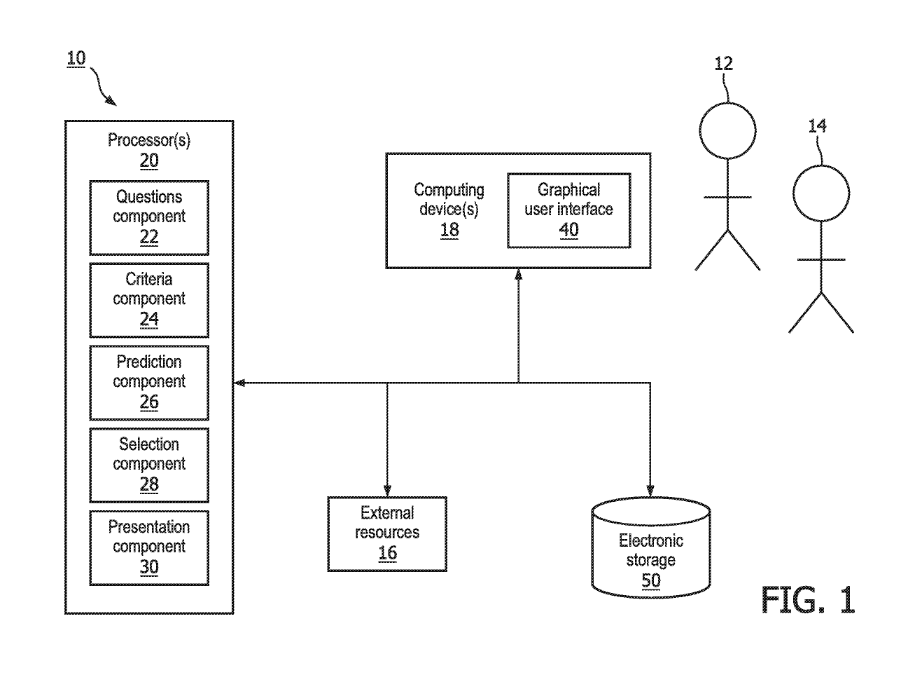

FIG. 1 illustrates an adaptive questionnaire generation system 10. Advantageously, system 10 is configured to generate adaptive questionnaires configured to facilitate diagnosis and/or treatment of medical conditions and/or other characteristics of a patient 12 such as depression, ADHD, movement ability, quality of life, sleep quality, and/or other conditions. System 10 is configured to obtain questions and corresponding answers posed to healthy individuals and those who were experiencing one or more medical conditions. This information is used as input to train a prediction model which, based on expectation criteria of a caregiver 14 (e.g., doctors, nurses, friends, family members, administrators, staff members, technicians, etc.), generates a questionnaire for patient 12. The expectation criteria include information related to a number of questions for the questionnaire, a quality metric threshold level that indicates a desired relatedness of the questionnaire to the medical condition(s) of the patient, and/or other information. If patient 12 decides to not answer one or more questions in the questionnaire for any reason, system 10 is configured such that one or more additional questions are asked of patient 12. The one or more additional questions are determined based on the previously answered questions by patient 12, the caregiver expectation criteria, classifiers for previously answered and/or the alternate additional questions (e.g., as described below), quality metric values for the previously answered and/or alternate additional questions (e.g., as described below), and/or based on other information.

In some embodiments, system 10 includes one or more of external resources 16, computing devices 18, processors 20, electronic storage 50, and/or other components.

External resources 16 include sources of possible questions, previously asked and answered question information, and/or other resources. Possible questions may include questions related to a medical condition of patient 12 and/or other patients, and/or other questions. The previously asked and answered question information includes individual questions and/or corresponding answers. In some embodiments, external resources 16 include sources of question information such as databases, websites, etc.; external entities participating with system 10 (e.g., a medical records system of a health care provider that stores medical history information for populations of patients), one or more servers outside of system 10, and/or other sources of information. In some embodiments, external resources 16 include components that facilitate communication of information such as a network (e.g., the internet), electronic storage, equipment related to Wi-Fi technology, equipment related to Bluetooth® technology, data entry devices, sensors, scanners, and/or other resources. For example, in some embodiments, external resources 16 may include a database where questions, and/or questions and corresponding answers are stored, and/or other sources of information. In some embodiments, the questions, and/or question and answer information comprises a subject matter of a given question, scoring and/or labeling for given questions (e.g., as described below), and/or other information. In some embodiments, some or all of the functionality attributed herein to external resources 16 may be provided by resources included in system 10. External resources 16 may be configured to communicate with processor 20, computing devices 18, electronic storage 50, and/or other components of system 10 via wired and/or wireless connections, via a network (e.g., a local area network and/or the internet), via cellular technology, via Wi-Fi technology, and/or via other resources.

Computing devices 18 are configured to provide interfaces between patients 12, caregivers 14, and/or other users, and system 10. In some embodiments, individual computing devices 18 are and/or are included in desktop computers, laptop computers, tablet computers, smartphones, and/or other computing devices associated with individual caregivers 14, individual patients 12, and/or other users. In some embodiments, individual computing devices 18 are, and/or are included in equipment used in hospitals, doctor's offices, and/or other medical facilities to monitor patients 12; test equipment; equipment for treating patients 12; data entry equipment; and/or other devices. Computing devices 18 are configured to provide information to and/or receive information from caregivers 14, patients 12, and/or other users. For example, computing devices 18 are configured to present a graphical user interface 40 to caregivers 14 to facilitate entry and/or selection of caregiver expectation criteria (e.g., as described below). In some embodiments, graphical user interface 40 includes a plurality of separate interfaces associated with computing devices 18, processor 20 and/or other components of system 10; multiple views and/or fields configured to convey information to and/or receive information from caregivers 14, patients 12, and/or other users; and/or other interfaces.

In some embodiments, computing devices 18 are configured to provide graphical user interface 40, processing capabilities, databases, electronic storage, and/or other resources to system 10. As such, computing devices 18 may include processors 20, electronic storage 50, external resources 16, and/or other components of system 10. In some embodiments, computing devices 18 are connected to a network (e.g., the internet). In some embodiments, computing devices 18 do not include processors 20, electronic storage 50, external resources 16, and/or other components of system 10, but instead communicate with these components via the network. The connection to the network may be wireless or wired. For example, processor 20 may be located in a remote server and may wirelessly cause display of graphical user interface 40 to a caregiver 14 on a computing device 18 associated with caregiver 14 and/or to a patient 12 on a computing device 18 associated with patient 12. As described above, in some embodiments, an individual computing device 18 is a laptop, a personal computer, a smartphone, a tablet computer, and/or other computing devices. Examples of interface devices suitable for inclusion in an individual computing device 18 include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that an individual computing device 18 includes a removable storage interface. In this example, information may be loaded into a computing device 18 from removable storage (e.g., a smart card, a flash drive, a removable disk) that enables the caregivers 14, patients 12, and/or other users to customize the implementation of computing devices 18. Other exemplary input devices and techniques adapted for use with computing devices 18 include, but are not limited to, an RS-232 port, RF link, an IR link, a modem (telephone, cable, etc.) and/or other devices.

Processor 20 is configured to provide information processing capabilities in system 10. As such, processor 20 may comprise one or more of a digital processor, an analog processor, a digital circuit designed to process information, an analog circuit designed to process information, a state machine, and/or other mechanisms for electronically processing information. Although processor 20 is shown in FIG. 1 as a single entity, this is for illustrative purposes only. In some embodiments, processor 20 may comprise a plurality of processing units. These processing units may be physically located within the same device (e.g., a server), or processor 20 may represent processing functionality of a plurality of devices operating in coordination (e.g., one or more servers, one or more computing devices 18 associated with a patient 12, caregivers 14, devices that are part of external resources 16, electronic storage 50, and/or other devices.)

In some embodiments, processor 20, external resources 16, computing devices 18, electronic storage 50, and/or other components may be operatively linked via one or more electronic communication links. For example, such electronic communication links may be established, at least in part, via a network such as the Internet, and/or other networks. It will be appreciated that this is not intended to be limiting, and that the scope of this disclosure includes embodiments in which these components may be operatively linked via some other communication media. In some embodiments, processor 20 is configured to communicate with external resources 16, computing devices 18, electronic storage 50, and/or other components according to a client/server architecture, a peer-to-peer architecture, and/or other architectures.

As shown in FIG. 1, processor 20 is configured via machine-readable instructions to execute one or more computer program components. The one or more computer program components may comprise one or more of a questions component 22, a criteria component 24, a prediction component 26, a selection component 28, a presentation component 30, and/or other components. Processor 20 may be configured to execute components 22, 24, 26, 28, and/or 30 by software; hardware; firmware; some combination of software, hardware, and/or firmware; and/or other mechanisms for configuring processing capabilities on processor 20.

It should be appreciated that although components 22, 24, 26, 28, and 30 are illustrated in FIG. 1 as being co-located within a single processing unit, in embodiments in which processor 20 comprises multiple processing units, one or more of components 22, 24, 26, 28, and/or 30 may be located remotely from the other components. The description of the functionality provided by the different components 22, 24, 26, 28, and/or 30 described below is for illustrative purposes, and is not intended to be limiting, as any of components 22, 24, 26, 28 and/or 30 may provide more or less functionality than is described. For example, one or more of components 22, 24, 26, 28, and/or 30 may be eliminated, and some or all of its functionality may be provided by other components 22, 24, 26, 28, and/or 30. As another example, processor 20 may be configured to execute one or more additional components that may perform some or all of the functionality attributed below to one of components 22, 24, 26, 28, and/or 30.

Questions component 22 is configured to obtain questions, questions and corresponding answers to the questions, and/or other information. In some embodiments, the obtaining includes electronically importing the questions and/or corresponding answers (e.g., from external resources 16), facilitating entry and/or selection of the questions and/or answers (e.g., via computing devices 18), uploading and/or downloading information, receiving emails, texts, and/or other communications, and/or other activities. In some embodiments, the questions and/or answers are related to various medical conditions experienced by a plurality of patients 12. In some embodiments, the questions and corresponding answers include questions answered by a plurality of healthy individuals and a plurality of individuals with medical conditions. In some embodiments, the questions and corresponding answers include questions answered by a plurality of individuals with medical conditions similar to and/or the same as a medical condition of a given patient 12 for whom system 10 generates a questionnaire.

In some embodiments, the questions and/or answers are stored in a database (e.g., such as an electronic database included in external resources 16) and/or other databases, and obtained by questions component 22 from the database. In some embodiments, the database of questions and/or answers to the questions comprises labeled and scored questionnaires answered by the plurality of healthy individuals and the plurality of individuals with a medical condition similar to or the same as the medical condition of patient 12. In some embodiments, questions component 22 is configured to label and score the questionnaires. In some embodiments, labeling and scoring comprises binary labels indicating whether or not a patient has a medical condition, risk scores provided to indicate the severity of the condition assessed by a caregiver 14, and/or other information. In some embodiments, the labelled and scored questionnaires with the corresponding answers comprise input for the prediction model (described below).

Criteria component 24 is configured to receive caregiver expectation criteria. In some embodiments, criteria component 24 is configured to facilitate entry and/or selection of caregiver expectation criteria via a graphical user interface 40 of a computing device 18 associated with a given caregiver and/or via other devices. The caregiver expectation criteria convey the expectations of a caregiver 14 for information that may be determined from a questionnaire that is to be answered by a patient 12. The questionnaire is related to a medical condition of a patient 12, vital signs of a patient 12, physical conditions and/or symptoms experienced by a patient 12, previous medical treatment provided to a patient 12, information in medical records related to the previous medical treatment provided to a patient 12, treatment outcomes, and/or other information. The caregiver expectation criteria comprise information related to a number of questions for the questionnaire, a quality metric threshold level that indicates a desired relatedness of the questionnaire to the medical condition of patient 12, and/or other information. In some embodiments, the quality metric threshold level may indicate the desired relatedness of each of the individual questions themselves in a given questionnaire. In some embodiments, the quality metric is Area Under the Receiver Operating Characteristic Curve (AUC), specificity, sensitivity, F1 score, and/or other quality metrics. In some embodiments, the quality metric threshold level is a minimum AUC threshold level, a threshold range of AUC levels, minimum specificity, sensitivity, F1 Score threshold levels, and/or other quality metric threshold levels.

Prediction component 26 is configured to determine sets of possible questions for forming a questionnaire for a given patient 12. The sets of questions are determined using a prediction model and/or other resources. In some embodiments, prediction component 26 is configured to provide the caregiver expectation criteria to the prediction model. In some embodiments, prediction component 26 is configured to train the prediction model using the answers to the previously posed questions in the question database. The previously asked questions and corresponding answers are provided to the prediction model to train the prediction model for generating the sets of questions based on the caregiver expectation criteria and/or other information. As described above, the database of previously posed and answered questions comprises labeled and scored questionnaires answered by a plurality of healthy individuals and a plurality of individuals with a medical condition similar to or the same as the medical condition of a patient 12. The sets of possible questions for forming the present questionnaire are determined from individual questions in the database. The sets of possible questions are determined such that each of the sets of possible questions satisfies the caregiver expectation criteria.

In some embodiments, prediction component 26 is configured to determine individual questions and/or sets of questions that do not satisfy the caregiver expectation criteria and are thus not available for questionnaires that are to be presented to patient 12. For example, prediction component 26 may determine that questions 1, 2, 4, and 10-30 in the database of previously asked and answered questions may be used in and/or as a set of possible questions that could be used as the questionnaire for patient 12. Prediction component 26 may also determine that questions 5, 8, 20, and 45 are unavailable for use based on the caregiver expectation criteria and/or other criteria.

Prediction component 26 is configured such that each of the sets of possible questions has classifiers indicating subject matter categories for questions in the respective set of possible questions, and one or more quality metric values that indicate relatedness of the respective set of questions to the medical condition. The classifiers may be obtained by training machine learning algorithms using labeled/scored questionnaires and considering the expectation criteria, for example. The one or more quality metric values are determined based on the classifiers and/or other information.

For example, criteria component 24 and/or prediction component 26 are configured to facilitate entry and/or selection of a physician's (e.g., a caregiver's) expectations for the design and performance (e.g., a range of quality metric threshold values, a number of questions, etc.) of the classifiers such that different sets of questions are identified based on the design and performance expectations. In some embodiments, the physician may require that the classifiers have an AUC of about 85%, or the physician may require that the classifiers have an AUC of about 0.7 to about 0.9 (this example is not intended to be limiting).

In some embodiments, the prediction model may be and/or include a neutral network that is trained and utilized for generating the sets of possible questions. As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neutral unit itself) may have a threshold function such that the signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

By way of a non-limiting example, in some embodiments, prediction component 26 is configured to receive the caregiver expectation criteria from a caregiver 14 for a questionnaire that is to be generated for a given patient 12. Prediction component 26 is configured to process the caregiver expectation criteria via the prediction model (e.g., by providing the caregiver expectation criteria as input to the trained (e.g., using the previously asked questions and corresponding answers) prediction model to cause the prediction model to generate one or more sets of possible questions that could form the questionnaire.

Selection component 28 is configured to select a set of questions for presentation to a patient 12. Selection component 28 is configured to select a set of questions from the one or more sets of possible questions determined by prediction component 26 and/or other questions. In some embodiments, selection component 28 is configured such that a set of questions having a fewest number of questions that satisfies the quality metric threshold level is selected from the sets of possible questions. In some embodiments, selection component 28 may select a set of questions that satisfies the quality metric threshold with more than a minimum number of questions (e.g., responsive to a caregiver specifying a number of questions the questionnaire should have and/or for other reasons).

In some embodiments, selection component 28 is configured to store the sets of possible questions determined by prediction component 26 in a database of possible questionnaires (e.g., electronic storage 50) and/or in other locations, and then select a set of questions for presentation to patient 12 from the sets of questions stored in the database. In some embodiments, the sets of possible questions are stored in such a database by classifier and/or with their corresponding quality metric values, and/or other information. For example, in some embodiments, selection component 28 selects a classifier and a corresponding set of questions determined by prediction component 26 and/or other components that satisfies the expectation criteria (e.g., the quality metric threshold level and/or a number of questions) of a physician (caregiver) with a minimum number of questions in the questionnaire.

In some embodiments, before selecting the set of questions for presentation to patient 12, selection component 28 is configured to facilitate review of one or more sets of questions from the sets of possible questions by caregiver 14 and/or other users. In such embodiments, selection component 28 is configured to facilitate entry and/or selection of refined caregiver expectation criteria and/or other information. The refined caregiver expectation criteria comprise indications of whether to select or not select a specific set of questions as the questionnaire, whether to include or exclude individual questions from the one or more sets of possible questions in the questionnaire, an adjusted quality metric threshold level, and/or other information. In some embodiments, the set of questions for presentation to the patient as a questionnaire is selected and/or adjusted based on the refined caregiver expectation criteria and/or other information.

For example, a physician (e.g., a caregiver) may decide that for a given patient 12, after reviewing a selected set of questions and/or one or more determined sets of possible questions, questions for the questionnaire should only be drawn from questions 1-20 in the previously asked and answered questions database. In addition the physician may adjust the quality metric threshold such that the AUC of the classifier is greater than 85% (e.g., which may affect which ones of questions 1-20 are selected for the questionnaire). In this example, selection component 28 may determine that the questionnaire is formed by questions 1-8, 10, and 13, which have a classifier with an AUC of 87%, even though a questionnaire that uses all twenty questions might have a classifier with an AUC of 90% (e.g., because the questionnaire with questions 1-8, 10, and 13 is the set of possible questions with the least number of questions that still meet the quality metric threshold level entered and/or selected by the physician). This example is not intended to be limiting.

Presentation component 30 is configured to cause the selected set of questions to be presented to patient 12. In some embodiments, presentation component 30 is configured to cause presentation of the selected set of questions to patient 12 via a graphical user interface 40 of a computing device 18 associated with patient 12 and/or on other computing devices. In some embodiments, the presentation comprises graphical, textual, or other representations of the questions; provision of question and/or answer fields in various views of graphical user interface 40; and/or other presentation. In some embodiments, presentation component 30 is configured to cause the selected set of questions and corresponding answers to be presented to caregiver 14 and/or other users. In some embodiments, presentation component 30 is configured to cause presentation of the selected set of questions and the corresponding answers to caregiver 14 via a graphical user interface 40 of a computing device 18 associated with caregiver 14 and/or on other computing devices. In some embodiments, the presentation comprises graphical, textual, or other representations of the questions; provision of question and/or answer fields in various views of graphical user interface 40; and/or other presentation.

In some embodiments, responsive to patient 12 not answering a given question, presentation component 30 is configured to determine one or more alternate additional questions for presentation to patient 12 based on previously answered questions, the caregiver expectation criteria, the refined caregiver expectation criteria, classifiers for the alternate additional questions, quality metric values for the alternate additional questions, and/or other information. In some embodiments, presentation component 30 may determine that the caregiver expectation criteria cannot be met with the one or more alternate additional questions. In such embodiments, presentation component 30 is configured to facilitate notification (e.g., via computing device 18) of the caregiver and entry and/or selection of new and/or amended caregiver expectation criteria (e.g., a different number of questions, a lower quality metric threshold level, etc.).

In some embodiments, presentation component 30 is configured to update the prediction model based on the answers to the questions by the patient, questions that the patient skips, and/or other information. In some embodiments, presentation component 30 is configured to present the selected set of questions, skipped questions, added questions, corresponding answers, and/or other information to caregiver 14 and/or other users (e.g., as described above). In some embodiments, presentation component 30 is configured to determine and/or present diagnosis and/or other information for patient 12. Determining the diagnosis information may include determining patterns and/or trends in the answers of patient 12, comparing the answers of patient 12 to answers of other patients with similar and/or the same medical conditions, and/or performing other operations. In some embodiments, the diagnosis information is determined based on answers to questions in the questionnaire, skipped questions, the previously posed and answered questions in the database, and/or other information. In some embodiments, presentation component 30 is configured to cause presentation of the diagnosis information with a diagnosis accuracy indicator, along with the answers to the questions in the questionnaire. In some embodiments, the diagnosis accuracy indicator may be and/or be related to a confidence interval, may be a percentage indicator (e.g., 90% accurate), may be a color coded indicator (e.g., a green color indicates higher accuracy and a red color indicates lower accuracy), and/or may be other diagnosis accuracy indicators.

Electronic storage 50 comprises electronic storage media that electronically stores information (e.g., the questions and answers stored in the database). The electronic storage media of electronic storage 50 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 50 may be (in whole or in part) a separate component within system 10, or electronic storage 50 may be provided (in whole or in part) integrally with one or more other components of system 10 (e.g., computing devices 18, processor 20, etc.). In some embodiments, electronic storage 50 may be located in a server together with processor 20, in a server that is part of external resources 16, in a computing device 18, and/or in other locations. Electronic storage 50 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 50 may store software algorithms, information determined by processor 20, information received via a computing device 18 and/or graphical user interface 40 and/or other external computing systems, information received from external resources 16, and/or other information that enables system 10 to function as described herein.

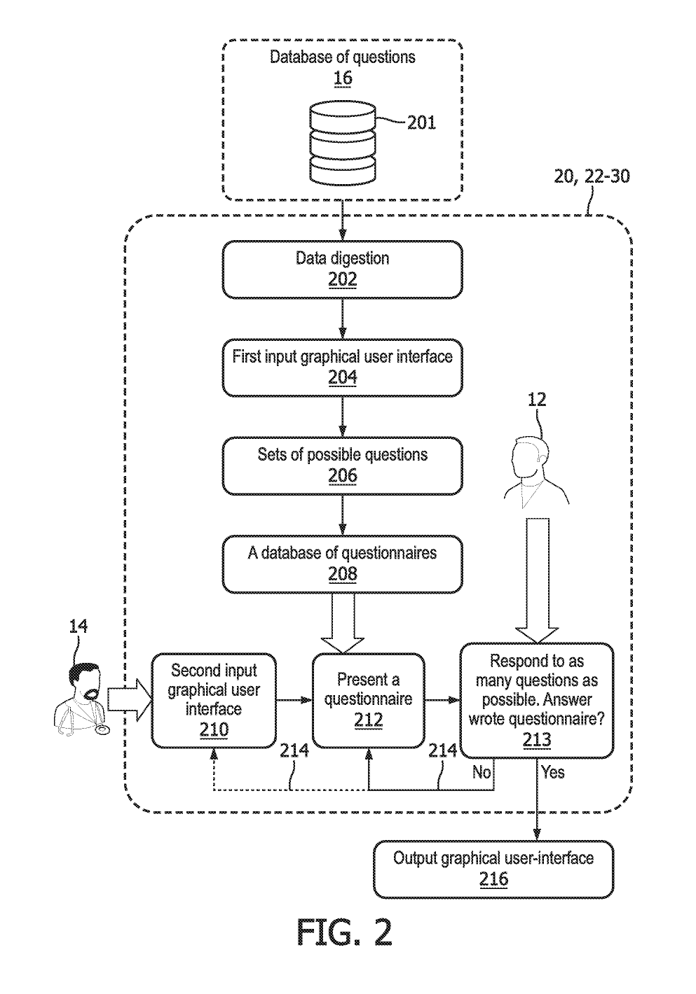

FIG. 2 illustrates examples of operations performed by system 10 (FIG. 1). At an operation 202, questions and/or corresponding answers to the questions are obtained (e.g., by questions component 22 of processor 20). In some embodiments, the questions and/or answers are stored in a database 201 (e.g., such as an electronic database included in external resources 16) and/or other databases. In some embodiments, the obtaining includes electronically importing the questions and corresponding answers, facilitating entry and/or selection of the questions and/or answers, uploading and/or downloading information, receiving emails, texts, and/or other communications, and/or other activities. In some embodiments, database 201 of posed and answered questions comprises labeled and scored questionnaires answered by a plurality of healthy individuals and a plurality of individuals with a medical condition similar to or the same as the medical condition of patient 12. In some embodiments, operation 202 includes labeling and scoring the questionnaires.

At an operation 204, first caregiver expectation criteria are received (e.g., by criteria component 24 of processor 20). The first caregiver expectation criteria convey the expectations of caregiver 14 for information that may be determined from a questionnaire to be answered by patient 12. As described herein, the caregiver expectation criteria comprise information related to a number of questions for the questionnaire, a quality metric threshold level that indicates a desired relatedness of the questionnaire to the medical condition, and/or other information. In some embodiments, operation 204 includes facilitating entry and/or selection of the first caregiver expectation criteria via a user interface (e.g., user interface 40 shown in FIG. 1) on a computing device associated with caregiver 14 (e.g., a computing device 18 shown in FIG. 1), and/or via other devices.

At an operation 206, sets of possible questions for forming the questionnaire are determined. The sets of questions are determined using a prediction model and/or other neural network and/or artificial intelligence resources. The sets of possible questions are determined such that each of the sets of possible questions satisfies the first caregiver expectation criteria. Each of the sets of possible questions has classifiers indicating subject matter categories for questions in the respective set of possible questions, and one or more quality metric values that indicate relatedness of the respective set of questions to the medical condition of patient 12. The one or more quality metric values are determined based on the classifiers and/or other information.

At an operation 208, the sets of possible questions are stored (e.g., by selection component 28 of processor 20 as described above) in a database of possible questionnaires (e.g., electronic storage 50) and/or in other locations. In some embodiments, the sets of possible questions are stored in such a database by classifier and/or with their corresponding quality metric values, and/or other information.

At an operation 210, a set of questions is selected (e.g., by selection component 28 of processor 20) for presentation to patient 12. A set of questions having a fewest number of questions that still satisfies the quality metric threshold level is selected from the sets of possible questions. Operation 210 includes facilitating review of the selected set and/or other sets of possible questions by caregiver 14, and entry and/or selection of second (e.g., refined) caregiver expectation criteria and/or other information. As described herein, the refined caregiver expectation criteria may comprise indications of whether to include or exclude specific questions in the questionnaire, an adjusted quality metric threshold level, and/or other information. In some embodiments, the set of questions is selected and/or adjusted based on the refined caregiver expectation criteria. In some embodiments, operation 210 includes facilitating entry and/or selection of the second caregiver expectation criteria via a user interface (e.g., user interface 40 shown in FIG. 1) on the computing device associated with caregiver 14 (e.g., a computing device 18 shown in FIG. 1), and/or via other devices.

At an operation 212, the selected set of questions is presented to patient 12 (e.g., by presentation component 30 of processor 20 via a user interface 40 on a computing device 18 associated with patient 12) as a questionnaire. At an operation 213, patient 12 answers the questions in the questionnaire. In some embodiments, responsive to patient 12 not answering a given question and/or questions, operation 212 includes determining 214 one or more alternate additional questions for presentation to patient 12 based on previously answered questions, the first and/or second caregiver expectation criteria, classifiers for the alternate additional questions, quality metric values for the alternate additional questions, and/or other information.

At an operation 216, the selected set of questions, skipped and/or otherwise unanswered questions, added questions, corresponding answers, and/or other information is presented to caregiver 14 and/or other users. In some embodiments, presentation component 30 of processor 20 is configured to cause presentation of the selected set of questions and the corresponding answers to caregiver 14 via the graphical user interface 40 (FIG. 1) of the computing device 18 (FIG. 1) associated with caregiver 14 and/or on other computing devices.

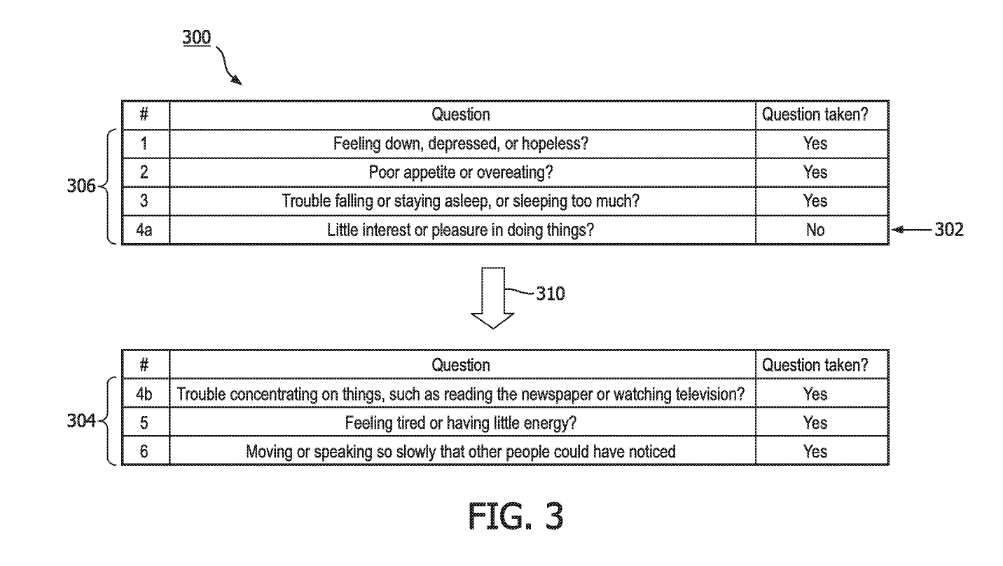

By way of a non-limiting example, FIG. 3 illustrates a skipped and/or otherwise unanswered questionnaire 300 question 302 (question number 4a in FIG. 3) and additional alternative questions 304 presented to a patient (e.g., patient 12 shown in FIGS. 1 and 2). As shown in FIG. 3, a set of questions 306 is presented to a patient (e.g., by presentation component 30 of processor 20 via a user interface 40 on a computing device 18 associated with patient 12 as described with respect to FIG. 1 above) as questionnaire 300. The patient answers questions 1-3 in questionnaire 300, but not question 4a. Responsive to the patient not answering question 4a, one or more alternate additional questions 304 (e.g., questions 4b, 5, and 6) are determined for presentation 310 to the patient. As described above, questions 304 may be determined based on previously answered questions 1-3, the caregiver expectation criteria, classifiers for the alternate additional questions, quality metric values for the alternate additional questions, and/or other information. In some embodiments, the operations illustrated by example in FIG. 3 may be repeated one or more times as a patient answers (or does not answer) questions in a given questionnaire.

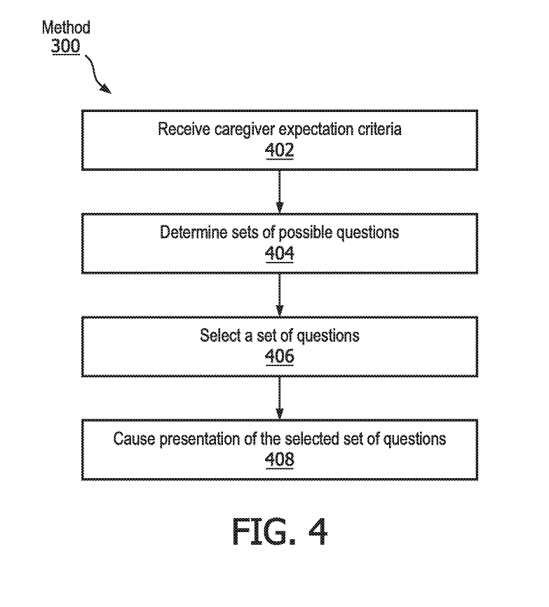

FIG. 4 illustrates a method 400 for generating an adaptive questionnaire with a generation system. The system comprises one or more hardware processors and/or other components. The one or more hardware processors are configured by machine readable instructions to execute computer program components. The computer program components include a questions component, a criteria component, a prediction component, a selection component, a presentation component, and/or other components. The operations of method 400 presented below are intended to be illustrative. In some embodiments, method 400 may be accomplished with one or more additional operations not described, and/or without one or more of the operations discussed. Additionally, the order in which the operations of method 400 are illustrated in FIG. 4 and described below is not intended to be limiting.

In some embodiments, method 400 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 400 in response to instructions stored electronically on an electronic storage medium. The one or more processing devices may include one or more devices configured through hardware, firmware, and/or software to be specifically designed for execution of one or more of the operations of method 400.

At an operation 402, caregiver expectation criteria are received. The caregiver expectation criteria convey the expectations of a caregiver for information that may be determined from a questionnaire answered by a patient. The questionnaire is related to a medical condition of the patient and/or other characteristics of the patient. The caregiver expectation criteria comprise information related to a number of questions for the questionnaire, a quality metric threshold level that indicates a desired relatedness of the questionnaire to the medical condition, and/or other information. In some embodiments, the quality metric is Area Under the Receiver Operating Characteristic Curve (AUC) and/or other quality metrics. In some embodiments, the quality metric threshold level is a minimum AUC threshold level, a threshold range of AUC levels and/or other quality metric threshold levels. In some embodiments, operation 402 is performed by a processor component the same as or similar to criteria component 24 (shown in FIG. 1 and described herein).

At an operation 404, sets of possible questions for forming the questionnaire are determined. The sets of questions are determined using a prediction model and/or other resources. The sets of possible questions are determined such that each of the sets of possible questions satisfies the caregiver expectation criteria. Each of the sets of possible questions has classifiers indicating subject matter categories for questions in the respective set of possible questions, and one or more quality metric values that indicate relatedness of the respective set of questions to the medical condition. The one or more quality metric values are determined based on the classifiers and/or other information. In some embodiments, operation 404 includes training the prediction model using answers to previously posed questions in a question database. The database of previously posed and answered questions comprises labeled and scored questionnaires answered by a plurality of healthy individuals and a plurality of individuals with a medical condition similar to or the same as the medical condition of the patient. The sets of possible questions for forming the questionnaire are determined from individual questions in the database. In some embodiments, operation 404 is performed by a processor component the same as or similar to prediction component 26 (shown in FIG. 1 and described herein).

At an operation 406, a set of questions is selected for presentation to the patient. A set of questions having a fewest number of questions that satisfies the quality metric threshold level is selected from the sets of possible questions. In some embodiments, operation 406 includes facilitating review of the sets of possible questions by the caregiver, and entry and/or selection of refined caregiver expectation criteria and/or other information. The refined caregiver expectation criteria comprise indications of whether to include or exclude specific questions in the questionnaire, an adjusted quality metric threshold level, and/or other information. In some embodiments, the set of questions is selected and/or adjusted based on the refined caregiver expectation criteria. In some embodiments, operation 406 is performed by a processor component the same as or similar to selection component 28 (shown in FIG. 1 and described herein).

At an operation 408, the selected set of questions is presented to a patient. In some embodiments, responsive to the patient not answering a given question, operation 408 includes determining one or more alternate additional questions for presentation to the patient based on previously answered questions, the caregiver expectation criteria, classifiers for the alternate additional questions, and quality metric values for the alternate additional questions. In some embodiments, operation 408 includes updating the prediction model based on the answers to the questions by the patient and questions that the patient skips. In some embodiments, operation 408 is caused by a processor component the same as or similar to presentation component 30 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. An adaptive questionnaire generation system, the system comprising one or more hardware processors configured by machine readable instructions to:
receive caregiver expectation criteria for a questionnaire related to a medical condition of a patient, the caregiver expectation criteria comprising (i) information related to a number of questions for the questionnaire and (ii) a quality metric threshold level that indicates a desired relatedness of the questionnaire to the medical condition;
use a prediction model to determine sets of possible questions for forming the questionnaire such that each of the sets of possible questions satisfies the caregiver expectation criteria, each of the sets of possible questions having:
(1) classifiers indicating subject matter categories for questions in the respective set of possible questions, and
(2) one or more quality metric values that indicate relatedness of the respective set of questions to the medical condition, the one or more quality metric values determined based on the classifiers;
select a set of questions having a fewest number of questions that satisfies the quality metric threshold level from the sets of possible questions for presentation to the patient; and
cause presentation of the selected set of questions to the patient.

2. The system of claim 1, wherein the one or more hardware processors are further configured to train the prediction model using answers to previously posed questions in a question database, the database of previously posed and answered questions comprising labeled and scored questionnaires answered by a plurality of healthy individuals and a plurality of individuals with a medical condition similar to or the same as the medical condition of the patient, wherein the sets of possible questions for forming the questionnaire are determined from individual questions in the database.

3. The system of claim 1, wherein the one or more hardware processors are configured to, responsive to the patient not answering a given question, determine one or more alternate additional questions for presentation to the patient based on previously answered questions, the caregiver expectation criteria, classifiers for the alternate additional questions, and quality metric values for the alternate additional questions, and
wherein the one or more processors update the prediction model based on (i) the answers to the questions by the patient and (ii) questions presented to but not answered by the patient.

4. The system of claim 1, wherein the one or more hardware processors are further configured to facilitate:
review of the sets of possible questions by the caregiver; and
entry and/or selection of refined caregiver expectation criteria, the refined caregiver expectation criteria comprising one or both of:
indications of whether to include or exclude specific questions in the questionnaire; and
an adjusted quality metric threshold level.

5. The system of claim 1, wherein the one or more hardware processors are configured such that the quality metric threshold level is indicative of Area Under the Receiver Operating Characteristic Curve (AUC).

6. The system of claim 5, wherein the one or more hardware processors are configured such that the quality metric threshold level is a minimum AUC threshold level or a threshold range of AUC levels.

7. A method for generating an adaptive questionnaire with a generation system, the system comprising one or more hardware processors configured by machine readable instructions, the method comprising:
receiving, with the one or more hardware processors, caregiver expectation criteria for a questionnaire related to a medical condition of a patient, the caregiver expectation criteria comprising (i) information related to a number of questions for the questionnaire and (ii) a quality metric threshold level that indicates a desired relatedness of the questionnaire to the medical condition;
using a prediction model to determine, with the one or more hardware processors, sets of possible questions for forming the questionnaire such that each of the sets of possible questions satisfies the caregiver expectation criteria, each of the sets of possible questions having:
(1) classifiers indicating subject matter categories for questions in the respective set of possible questions, and
(2) one or more quality metric values that indicate relatedness of the respective set of questions to the medical condition, the one or more quality metric values determined based on the classifiers;
selecting, with the one or more hardware processors, a set of questions having a fewest number of questions that satisfies the quality metric threshold level from the sets of possible questions for presentation to the patient; and
causing, with the one or more hardware processors, presentation of the selected set of questions to the patient.

8. The method of claim 7, wherein the method further comprises training the prediction model using answers to previously posed questions in a question database, the database of previously posed and answered questions comprising labeled and scored questionnaires answered by a plurality of healthy individuals and a plurality of individuals with a medical condition similar to or the same as the medical condition of the patient, wherein the sets of possible questions for forming the questionnaire are determined from individual questions in the database.

9. The method of claim 7, wherein the method further comprises, responsive to the patient not answering a given question, determining, with the one or more hardware processors, one or more alternate additional questions for presentation to the patient based on previously answered questions, the caregiver expectation criteria, classifiers for the alternate additional questions, and quality metric values for the alternate additional questions, and
wherein the prediction model is updated based on (i) the answers to the questions by the patient and (ii) questions presented to but not answered by the patient.

10. The method of claim 7, wherein the method further comprises facilitating, with the one or more hardware processors:
review of the sets of possible questions by the caregiver; and
entry and/or selection of refined caregiver expectation criteria, the refined caregiver expectation criteria comprising one or both of:
indications of whether to include or exclude specific questions in the questionnaire; and
an adjusted quality metric threshold level.

11. The method of claim 7, wherein the quality metric threshold level is indicative of Area Under the Receiver Operating Characteristic Curve (AUC).

12. The method of claim 11, wherein the quality metric threshold level is a minimum AUC threshold level or a threshold range of AUC levels.

13. A system for generating an adaptive questionnaire, the system comprising:
means for receiving caregiver expectation criteria for a questionnaire related to a medical condition of a patient, the caregiver expectation criteria comprising (i) information related to a number of questions for the questionnaire and (ii) a quality metric threshold level that indicates a desired relatedness of the questionnaire to the medical condition;
means for using a prediction model to determine sets of possible questions for forming the questionnaire such that each of the sets of possible questions satisfies the caregiver expectation criteria, each of the sets of possible questions having:
(1) classifiers indicating subject matter categories for questions in the respective set of possible questions, and
(2) one or more quality metric values that indicate relatedness of the respective set of questions to the medical condition, the one or more quality metric values determined based on the classifiers;
means for selecting a set of questions having a fewest number of questions that satisfies the quality metric threshold level from the sets of possible questions for presentation to the patient; and
means for causing presentation of the selected set of questions to the patient.

14. The system of claim 13, further comprising means for training the prediction model using answers to previously posed questions in a question database, the database of previously posed and answered questions comprising labeled and scored questionnaires answered by a plurality of healthy individuals and a plurality of individuals with a medical condition similar to or the same as the medical condition of the patient, wherein the sets of possible questions for forming the questionnaire are determined from individual questions in the database.

15. The system of claim 13, further comprising means for, responsive to the patient not answering a given question, determining one or more alternate additional questions for presentation to the patient based on previously answered questions, the caregiver expectation criteria, classifiers for the alternate additional questions, and quality metric values for the alternate additional questions, and
wherein the prediction model is updated based on (i) the answers to the questions by the patient and (ii) questions presented to but not answered by the patient.

16. The system of claim 13, further comprising means for facilitating:
review of the sets of possible questions by the caregiver; and
entry and/or selection of refined caregiver expectation criteria, the refined caregiver expectation criteria comprising one or both of:
indications of whether to include or exclude specific questions in the questionnaire; and
an adjusted quality metric threshold level.

17. The system of claim 13, wherein the quality metric threshold level is indicative of Area Under the Receiver Operating Characteristic Curve (AUC).

18. The system of claim 17, wherein the quality metric threshold level is a minimum AUC threshold level or a threshold range of AUC levels.
